(19) United States
(12) Patent Application Publication
(10) Pub. No.: US 2021/0188741 A1
(43) Pub. Date: Jun. 24, 2021

(54) OXIDATIVE DEHYDROGENATION OF ALKANES TO ALKENES USING SULFUR AS AN OXIDANT

(71) Applicant: Northwestern University, Evanston, IL (US)

(72) Inventors: Tobin Jay Marks, Evanston, IL (US); Tracy L. Lohr, Houston, TX (US); Shanfu Liu, Chicago, IL (US); Allison Marie Arinaga, Bethesda, MD (US)

(21) Appl. No.: 17/173,663

(22) Filed: Feb. 11, 2021

Related U.S. Application Data
(63) Continuation-in-part of application No. PCT/US19/46286, filed on Aug. 13, 2019.
(60) Provisional application No. 62/718,052, filed on Aug. 13, 2018.

(51) Int. Cl. C07C 5/46; B01J 23/22; B01J 21/04; B01J 27/04

(52) U.S. Cl. CPC C07C 5/46; B01J 23/22; B01J 21/04; C07C 2527/04; C07C 2523/22; C07C 2521/04; B01J 27/04

(57) ABSTRACT

The present disclosure provides a method for the oxidative dehydrogenation of an alkane, e.g., ethane, propane, etc. In embodiments, a method for oxidative dehydrogenation of an alkane comprises exposing a gas comprising an alkane having 2 or more carbons to elemental sulfur vapor at an elevated reaction temperature and for a period of time to convert the alkane to one or more products via oxidative dehydrogenation, the one or more products comprising a primary alkene.

### CROSS REFERENCE TO RELATED APPLICATIONS

The present application is a continuation-in-part of International patent application number PCT/US19/46286, which was filed on Aug. 13, 2019, the entire contents of which are hereby incorporated by reference, which claims priority from U.S. provisional patent application No. 62/718,052, which was filed on Aug. 13, 2018, the entire contents of which are hereby incorporated by reference.

### REFERENCE TO GOVERNMENT RIGHTS

This invention was made with government support under 1647722 awarded by the National Science Foundation. The government has certain rights in the invention.

### BACKGROUND

The direct, pyrolytic production of ethylene from ethane is extremely energetically costly and consumes ˜19% of the total energy consumption required for the production of all commodity chemicals worldwide. Producing ethylene by the oxidative dehydrogenation of ethane (ODHE) could lower the energetic costs by 35˜54%. However, rapid coking on catalyst surfaces deactivates ODHE catalysts in many systems. The intensive use of costly catalysts by the ODHE process has also hindered its industrial application. Moreover, the by-products of ODHE, CO and CO₂, are not eco-friendly nor industrially of current critical need, and often require post-treatment (i.e., CO oxidation to CO₂ and subsequent capture).

### SUMMARY

The present disclosure provides a method for the oxidative dehydrogenation of alkanes, e.g., ethane, propane, etc. The method is based on using elemental sulfur as the oxidant. Exemplified with the alkane ethane, this revolutionary process, ODHE using sulfur (SODHE), is able to provide an excellent ethylene yield of over 70%, on par with the best ODHE catalysts, and exceeds the yields of standard industry pyrolysis. The conversion of ethane to ethylene may take place entirely in the gas phase without requiring a noble metal catalyst, and operates with a great variety of inexpensive, earth abundant and non-toxic oxide catalysts, which contributes to the simplicity and low cost of industrial implementation of this process. Impressive results are also achieved for oxidative dehydrogenation of propane using elemental sulfur and a sulfided V/Al₂O₃ catalyst.

In embodiments, a method for oxidative dehydrogenation of an alkane comprises exposing a gas comprising an alkane having 2 or more carbons to elemental sulfur vapor at an elevated reaction temperature and for a period of time to convert the alkane to one or more products via oxidative dehydrogenation, the one or more products comprising a primary alkene.

Other principal features and advantages of the present disclosure will become apparent to those skilled in the art upon review of the following drawings, the detailed description, and the appended claims.

### BRIEF DESCRIPTION OF THE DRAWINGS

Illustrative embodiments of the present disclosure will hereafter be described with reference to the accompanying drawings.

FIG. 1 shows a schematic diagram of the design for a custom plug-flow reactor for the oxidative dehydrogenation of alkanes, e.g., ethane, according to an illustrative embodiment.

FIGS. 2A-2D show catalyst performance as a function of reaction temperature in the oxidative dehydrogenation of ethane according to an illustrative embodiment.

FIG. 3 shows conversion and selectivity in the oxidative dehydrogenation of ethane using Cr₂O₃ (precatalyst) as a function of time on stream at 940° C.

FIGS. 4A-4D show product distribution as a function of reaction temperature in the oxidative dehydrogenation of ethane. The order of the products in the legend matches the order of the products shown in the bars at each temperature. For example, for the last bar near 950° C., CH₄ is at the bottom, followed by C₂H₄, followed by C₂H₂, followed by CS₂ at the top.

FIG. 5 shows product distribution and conversion as a function of reaction temperature in the oxidative dehydrogenation of propane. The order of the products in the legend matches the order of the products shown in the bars at each temperature. Thus, for example, for the last bar at 950° C., CH₄ is at the bottom, followed by C₂H₄, followed by C₂H₂, followed by C₂H₆, followed by C₃H₆ at the top.

FIGS. 6A-6B show conversion (FIG. 6A) and selectivity (FIG. 6B) in the oxidative dehydrogenation of propane using various precatalysts and blank controls as a function of temperature.

FIGS. 7A-7C show product distribution as a function of temperature for different precatalysts (or blank controls) in the oxidative dehydrogenation of propane. The order of the products in the legend matches the order of the products shown in the bars at each temperature. For example, for the last bar at the right, CH₄ is at the bottom, followed by C₂H₄, followed by C₂H₆, followed by C₂H₂, followed by CS₂, followed by C₃H₆ at the top.

FIG. 8 shows propylene selectivity as a function of propane conversion using a sulfided bulk ZrO₂ catalyst and elemental sulfur (see Example 3); existing metal oxide catalysts and O₂; and a sulfided V/Al₂O₃ catalyst and elemental sulfur (see Example 4).

### DETAILED DESCRIPTION

In embodiments, a method for the oxidative dehydrogenation of an alkane comprises exposing a gas comprising an alkane to elemental sulfur vapor at an elevated reaction temperature to convert the alkane to one or more products via oxidative dehydrogenation. The gas comprising the alkane may also comprise one or more inert gases (e.g., helium, argon, etc.). By “elemental sulfur vapor” is meant a gas comprising S₂, although the elemental sulfur vapor may comprise other sulfur allotropes. However, at least in embodiments, no other sulfur-containing compound is significantly present in the elemental sulfur vapor. Elemental sulfur vapor may be generated by heating solid sulfur (S₈) as described in the Examples, below. By “elevated reaction temperature” it is meant greater than room temperature (20-25° C.). The specific temperature may be selected to provide a desired (e.g., maximum) conversion of the alkane and/or a desired (e.g., maximum) yield/selectivity of a particular product. Illustrative elevated reaction temperatures include at least 200° C., at least 400° C., at least 500° C., in the range of from 200° C. to 2500° C., from 400° C. to 2000° C., from 500° C. to 1500° C., or from 600° C. to 900° C.

The method may be used with a variety of alkanes to provide a primary alkene (among other possible products). By “primary alkene” it is meant the alkene corresponding to the same number of carbons as the reactant alkane. Another possible product is a primary alkyne (primary has an analogous meaning). Products having fewer numbers of carbons than the reactant alkane may be produced. In embodiments, the alkane has 2, 3, 4, 5, 6, 10, 14, or 18 carbons. In embodiments, the alkane is ethane. In embodiments, the alkane is propane. In embodiments, the alkane is not methane and the method does not involve use of methane.

Other conditions under which the alkane is exposed to sulfur include the weight hourly space velocity (WHSV) of the alkane, the ratio of sulfur:alkane and the pressure. Again, the specific values may be selected to provide a desired (e.g., maximum) conversion of the alkane and/or a desired (e.g., maximum) yield/selectivity of a particular product. Using ethane by way of example, the WHSV of the ethane may be in the range of from 0.00523 to 10.46 h⁻¹, from 0.0523 h⁻¹ to 2.616 h⁻¹, or from 0.105 h⁻¹ to 0.785 h⁻¹. The sulfur:ethane ratio may be in the range of from 0 to 100, from 0.1 to 20, or from 0.1 to 5. The pressure may be in the range of from 0.01 psi to 200 psi, from 1 psi to 40 psi, from 2 psi to 5 psi, or from 2 psi to 4 psi. These ranges may also be used for other alkanes. Specific, illustrative values for propane as the alkane are provided in the Examples, below.

The exposure of the alkane to sulfur may take place entirely in the gas phase and in the absence of any catalyst. However, in embodiments, the exposure may take place in the presence of a catalyst. The catalyst may be one which is formed in situ, by exposing a precatalyst to a gas comprising S₂ and H₂S at an elevated temperature and for an activation time. The S₂/H₂S containing gas may also comprise other sulfur allotropes. However, at least in embodiments, no other sulfur-containing compound is present in the S₂/H₂S containing gas.

A variety of precatalysts may be used. Non-limiting, illustrative precatalysts include sulfides, oxides, oxysulfides of an alkali metal, e.g., Li₂O; alkaline earth metal oxides, e.g., MgO; redox active transition metal oxides, e.g., Cr₂O₃, Fe₃O₄, Co₂O₃; and late transition metal oxides, e.g., ZnO. The metallic state of an alkali metal, an alkaline earth metal, and a transition metal may also be used. Noble metals (e.g., Pt, Pd, Ag, etc.) and their oxides, oxysulfides, and sulfides may be used. Combinations of different types of precatalysts may be used. The precatalysts may be in nanoparticle form, and optionally located on a high surface area support.

As noted above, the precatalyst may be a supported transition metal oxide. In embodiments, the transition metal oxide is vanadium oxide (VOx). In embodiments, the high surface area support is a metal oxide support. The metal oxide of such a support may have formula MₓO_{y}, where M is be a metal or metalloid from Groups 1-5 or 12-14 of the Periodic Table (e.g., MgO, La₂O₃, TiO₂, Nb₂O₅, ZnO, Al₂O₃, or SiO₂), and having a surface area between 50 and 1000 m²/g. In embodiments, the metal oxide is alumina (Al₂O₃). The amount of the transition metal oxide (e.g., vanadium oxide) on the support (e.g., alumina) may be selected to control the nature of active sites on the surface of the support. The VOx sites may consist of isolated, polymerized, or vanadium oxide sites. Illustrative amounts include those in a range of from 0.1 weight % to 10 weight %, from 0.5 weight % to 10 weight %, and from 0.1 weight % to 5 weight %.

The elevated temperature to generate the catalyst from the precatalyst may be those described above. The activation time may be in the range of from 10 minutes to 20 hours, from 1 hr to 10 hrs, or from 2 hrs to 6 hrs. The exposure of the precatalyst may involve heating to the elevated reaction temperature (the temperature at which the alkane is exposed to the elemental sulfur vapor) over the activation time.

Exposure of certain precatalysts to the gas comprising S₂ and H₂S at the elevated temperature and for the activation time provides catalysts comprising compounds of formula MₓO_{y}S_{z}, wherein M is an alkali metal, an alkaline earth metal, or a transition metal and x>0, y≥0, and z≥0. In embodiments, M is a transition metal (e.g., V). As noted above, if the precatalyst is a supported precatalyst, the catalyst also comprises the high surface area support (e.g., alumina).

The alkane-containing gas and the elemental sulfur vapor may be considered to form a gaseous reactant mixture. If a precatalyst/catalyst is used, the gaseous reactant mixture may further comprise the S₂/H₂S from the activation of the precatalyst. However, at least in embodiments, the gases used to form the gaseous reactant mixture as well as the gaseous reactant mixture itself are free of O₂ and any oxygen-containing compound (however, this does not preclude the use of an oxygen-containing solid catalyst, e.g., an oxide catalyst). Similarly, at least in embodiments, the gases used to form the gaseous reactant mixture as well as the gaseous reactant mixture itself are free of any other sulfur-containing compound (i.e., only S₂ and optionally, a sulfur allotrope or H₂S are present). By “free” it is meant that the amount is zero or sufficiently close to zero so as not to have a material effect on the oxidative dehydrogenation reaction.

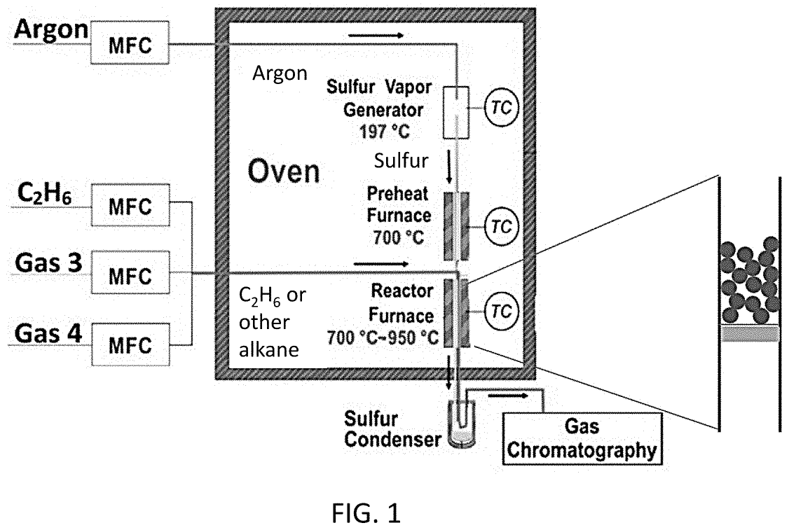

The method may be carried out using a variety of reactor systems. A suitable reactor system is the plug-flow reactor system shown in FIG. 1, which is further described in the Examples, below.

As further described in the Examples below, in at least in some embodiments, the method is able to achieve high values of alkane conversion. In embodiments, the alkane conversion is at least 80%, at least 85%, at least 90%, at least 95%, at least 99%, or about 100%. Alkane conversion is defined in the Examples, below. Similarly, at least in some embodiments, the method is able to achieve high selectivities, e.g., of a particular oxidative dehydrogenation product. By way of illustration, in embodiments, the method achieves a selectivity of a primary alkene or a primary alkyne of at least 50%, at least 55%, at least 60%, at least 65%, at least 70%, at least 75%, at least 80%, at least 85%, or at least 90%. Product selectivity may be defined as provided in the Examples, below. In embodiments, the conversions and selectivities in this paragraph refer to the conversion of ethane (e.g., to ethylene) or the conversion of propane (e.g., to propylene and to ethylene).

The conversion/selectivities described above may refer to a particular set of conditions, a reaction temperature at any value or range of values disclosed herein, alkane WHSV at any value or range of values disclosed herein, sulfur:alkane ratio at any value or range of values disclosed herein, presence/absence of a catalyst, and a pressure at any value or range of values disclosed herein. The conversion/selectivities may refer to a specific combination of these parameters as used in the Examples, below.

At least in some embodiments, the dehydrogenation process exhibits high stability, i.e., the conversion and/or selectivity values are constant over a period of time. By “constant,” it is meant that the values do not change by more than ±10%. The period of time may be at least 100 hours.

### EXAMPLES

### Example 1

This Example demonstrates the oxidative dehydrogenation of ethane using sulfur (SODHE) in which ethane is selectively oxidized to ethylene by elemental sulfur at elevated temperature. SODHE uses ethane and sulfur vapor with an inert carrier gas as the feed, and produces ethylene, along with H₂S, acetylene, CS₂ and trace amounts of propane and propylene. The reaction is operated at 940° C. Fe₃O₄, Cr₂O₃, and MgO have been tested as precatalysts for this reaction. The active catalysts are generated in situ during a sulfurization process. Under reaction conditions, the conversion of ethane is 99%, and the selectivity and yield of ethylene is approximately 70%. The catalysts have been tested for stability and the performance does not change for 60 hours on stream. Optionally, similar ethylene yields, ethane conversion and ethylene selectivities are observed using the quartz reactor.

Materials and Methods

The Fe₃O₄ and Cr₂O₃ nanopowders were purchased from Alfa Aesar with a purity of ≥97%. The MgO nanopowder was purchased from Sigma Aldrich with a purity of ≥97%. Reactor measurements were carried out in a custom packed bed reactor. The experimental set-up is shown in FIG. 1. The main body of the reactor is housed in a temperature-controlled oven to prevent sulfur condensation. The reactor consists of three major components: (1) sulfur vapor generator and preheat furnace that vaporizes and converts the solid sulfur phase S₈ to gaseous sulfur, principally S₂, (2) catalytic reaction furnace, and (3) on-stream analytical detection system. Regarding (1), the sulfur vapor generator generates a sulfur vapor, which consists of a variety of sulfur allotropes, ranging from S_{z} to S₈. The mixture of allotropes is heated in the preheat furnace to form a homogeneous S₂ vapor. Reactants and inert gas mixtures (Airgas) are introduced into the reactor with Brooks mass flow controllers. Prior to the reactor studies, the quartz reactor tube was charged with 200 mg of precatalyst (or no catalyst) with a particle size 180 μm-300 μm. During heating to T=950° C. and holding for 4 hours, the precatalyst was exposed to 0.28% S₂ and 0.33% H₂S before exposure to the reaction mixture of ethane, inert gas and S₂ vapor. The flow rates of hydrocarbon gases and the balance gases Ar and He were controlled with Brooks Model 5850E mass flow controllers. 4.97% C₂H₆/He was used in reactor measurements. The effluent distribution was continuously monitored by gas chromatography (Agilent 7890A, equipped with FID, TCD, and FPD detector).

### Results and Discussion

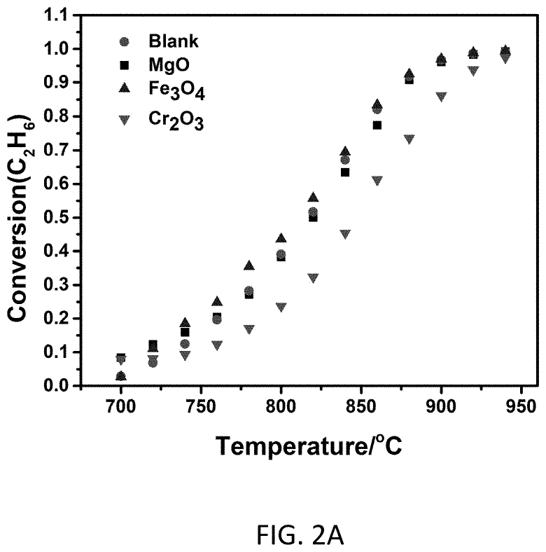

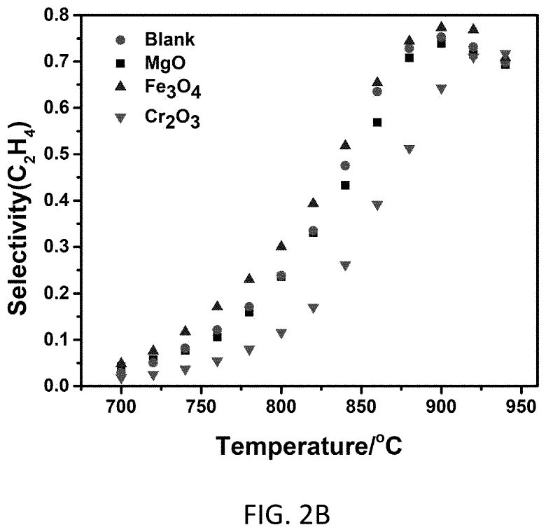

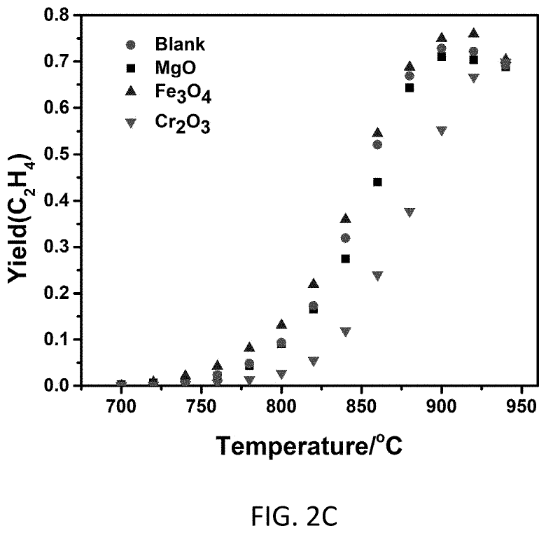

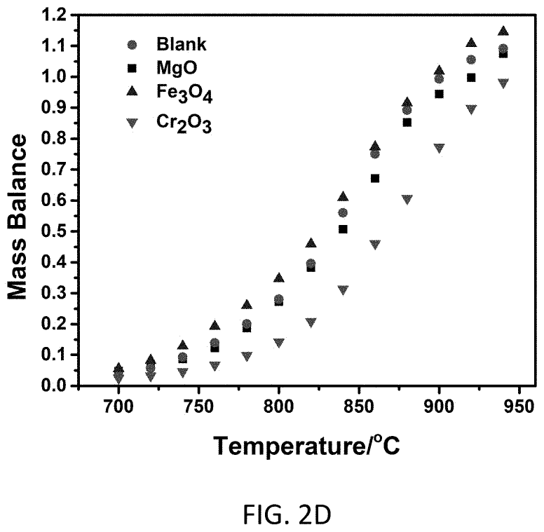

During preliminary experiments, the optimal operating temperature was determined for Fe₃O₄ MgO and Cr₂O₃ catalysts as well as the quartz control. These experiments were carried out at WHSV (weight hourly space velocity) of 0.628 h⁻¹ and C₂H₆: S₂ ratio=3.07. The conversion, selectivity, yield and mass balance are shown in FIGS. 2A-2D, respectively.

The conversion, selectivity, and yield are calculated based on the conservation of mass of carbon where CₓH_{y}S_{z} is ethylene, acetylene or carbon disulfide:

$$\begin{matrix}{C}_{2} {H}_{6} Conversion = \frac{{C}_{2} {H}_{{6}_{( inlet )}} - {C}_{2} {H}_{{6}_{( outlet )}}}{{C}_{2} {H}_{{6}_{( inlet )}}} & ( 1 ) \\ {C}_{2} {H}_{4} Selectivity = \frac{{C}_{2} {H}_{{4}_{( outlet )}}}{{C}_{2} {H}_{{6}_{( inlet )}} - {C}_{2} {H}_{{6}_{( outlet )}}} & ( 2 ) \\ {C}_{2} {H}_{4} Yield = {C}_{2} {H}_{6} Conversion \times {C}_{2} {H}_{4} Selectivity & ( 3 ) \\ Mass Balance = Σ {C}_{x} {H}_{y} {S}_{z} Selectivity & ( 4 )\end{matrix}$$

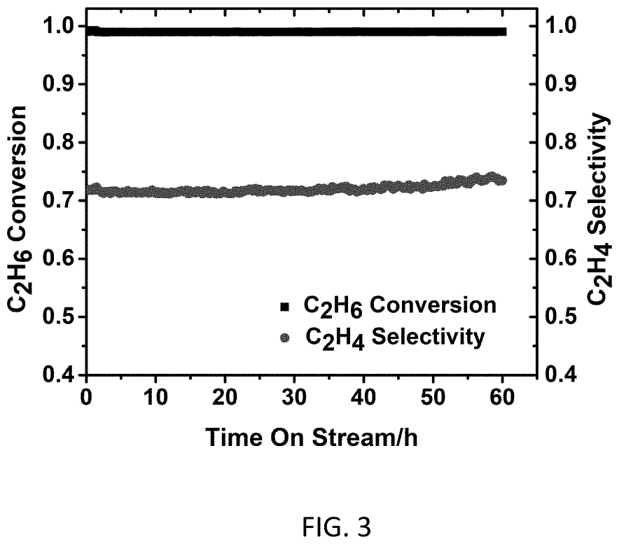

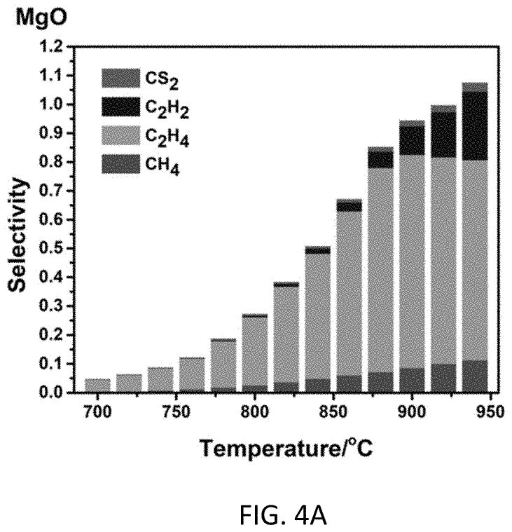

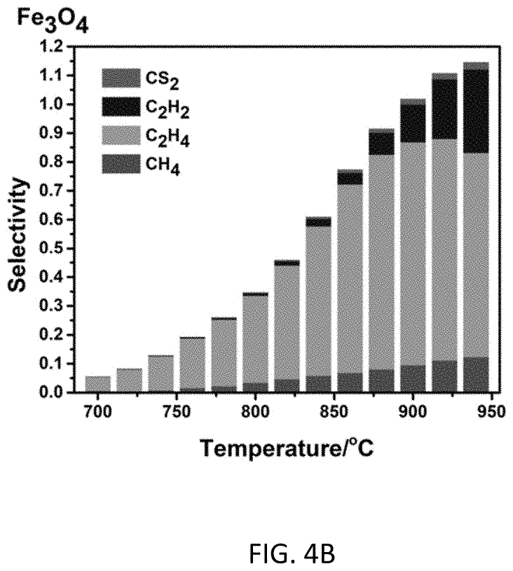

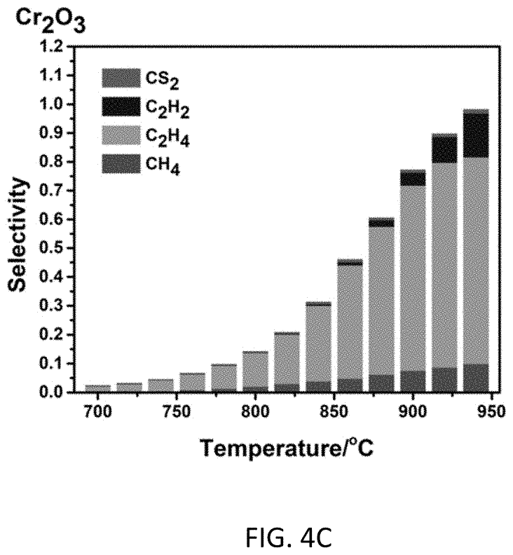

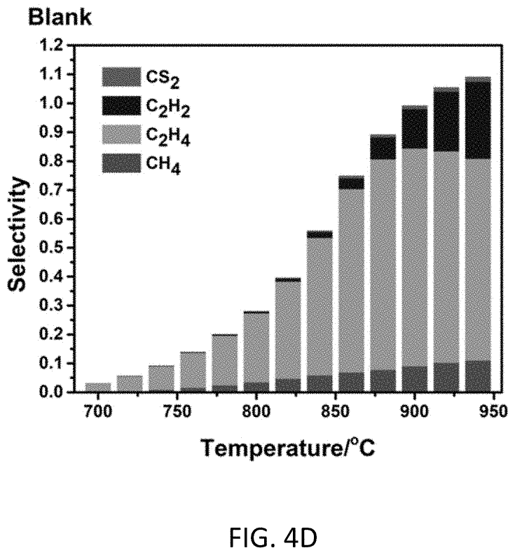

As seen from the FIG. 2C, ethylene yields can be achieved at over 70% for all catalysts at 940° C. Moreover, under these conditions, the selectivity and conversion are not significantly different on the different catalysts. The mass balance was also calculated for the 3 catalysts and the blank reaction. The mass balances at 940° C. are all near 100% within the margin of error. The conversion and selectivity of SODHE both increase with temperature. The thermal stability was tested for Cr₂O₃ at 940° C. for 60 hours. As shown in the example in FIG. 3, the catalytic performance remains constant for at least 60 hours. FIGS. 4A-4D provide a more comprehensive view of catalyst performance with selectivity to each carbon-containing species.

### Example 2

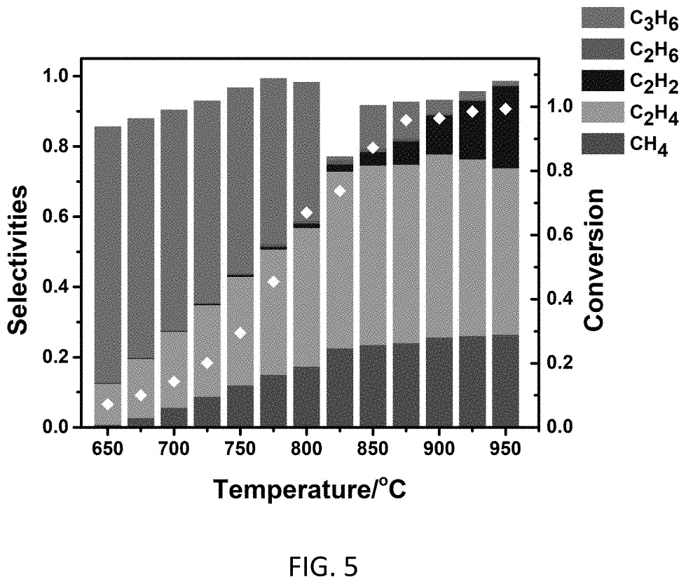

Experiments similar to those described above for Example 1 were conducted using propane as the reactant. The feed gas was propane and sulfur vapor, with a sulfur:propane ratio of 0.326 and a propane WHSV of 0.921 h⁻¹. The precatalyst was Fe₃O₄ nanopowder (purchased from Alfa Aesar with a purity of ≥97%). The reaction temperature ranged from 650° C. to 950° C., although lower temperatures may be used, e.g., 400° C. The products of this reaction included methane, ethane, ethylene, acetylene and propylene. The results are shown in FIG. 5.

As can be seen from FIG. 5, 99% of conversion of propane is achieved at 950° C., with moderate selectivity to ethylene. Up to 73% selectivity to propylene can be achieved at 650° C. and up to 50% selectivity can be achieved over a large temperature range (825° C. to 925° C.). As both ethylene and propylene are valuable commodity chemicals, the oxidative dehydrogenation of propane is an extremely useful reaction.

### Example 3

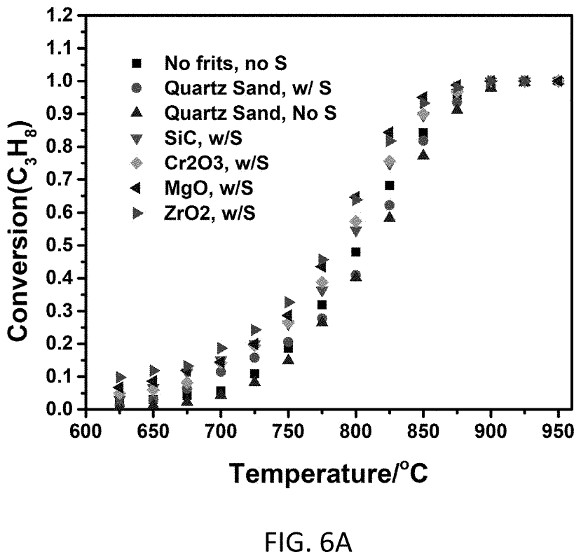

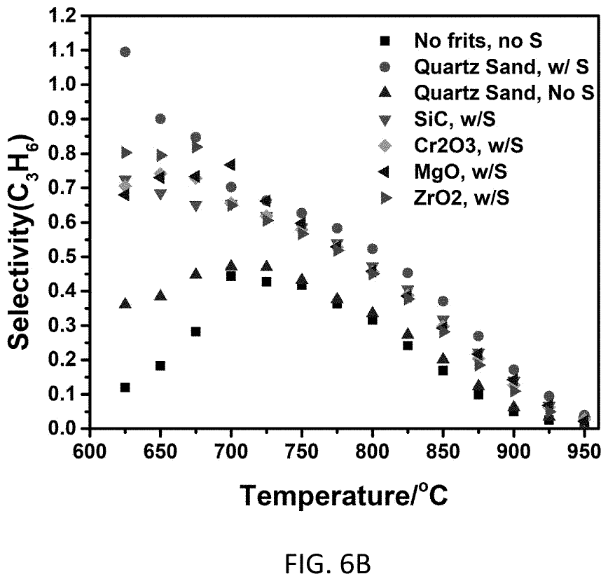

Additional experiments similar to those described above for Example 2 were conducted, again, using propane as the reactant. The feed gas was propane and sulfur vapor, with a sulfur:propane ratio of 0.326 and a propane WHSV of 0.628 h⁻¹. Various precatalysts (Cr₂O₃, w/S; MgO, w/S; ZrO₂, w/S) were used, including blank controls (No frits, no S; Quartz sand, w/S; Quartz sand, No S; SiC, w/S.). The term “w/S” means with S₂ which means that the precatalyst is exposed to S₂/H₂S and the reaction mixture is exposed to S₂. The term “no S” means without S₂ which means that the precatalyst is exposed to S₂/H₂S, but the reaction mixture is not exposed to S₂. The conversion and selectivity results are shown in FIGS. 6A and 6B, respectively. These results suggest that the activation of propane doesn't appear to be affected by surface acidity and redox activity. This is concluded from the fact that the conversion does not change significantly when different precatalysts are used, and the conversions are very similar to those on the blank controls. The results also show that S₂ greatly improves the selectivity for C₃H₆, although the precatalysts are not very effective at higher temperatures. This is concluded by comparing the selectivity of the precatalysts when sulfur is present vs. not present.

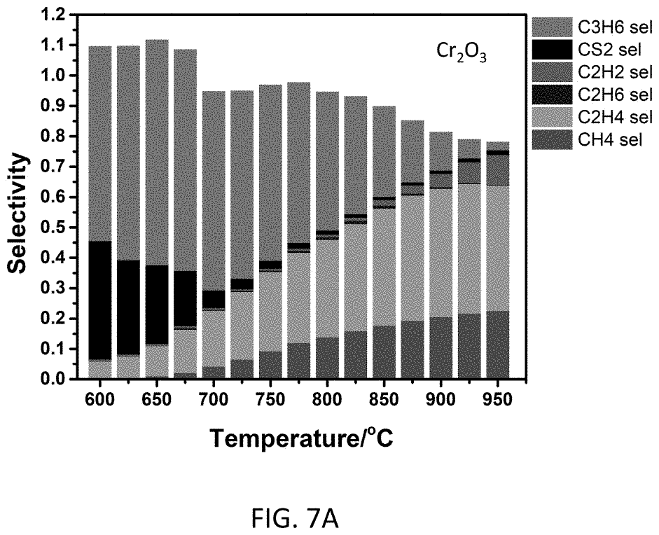

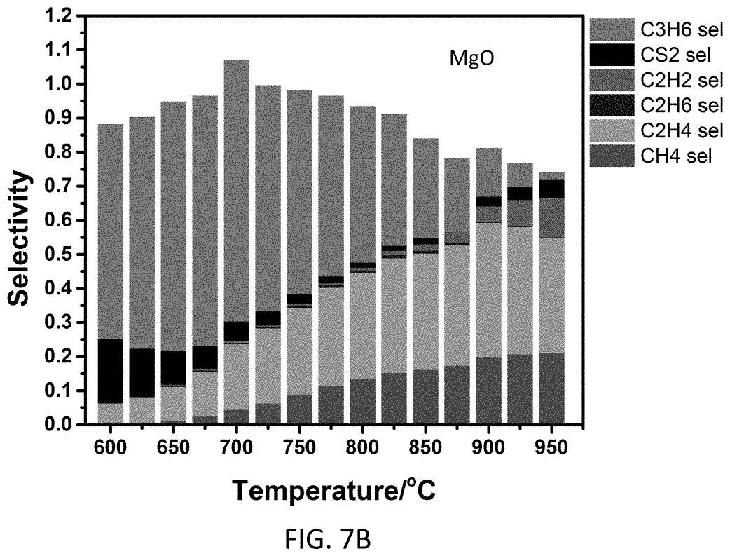

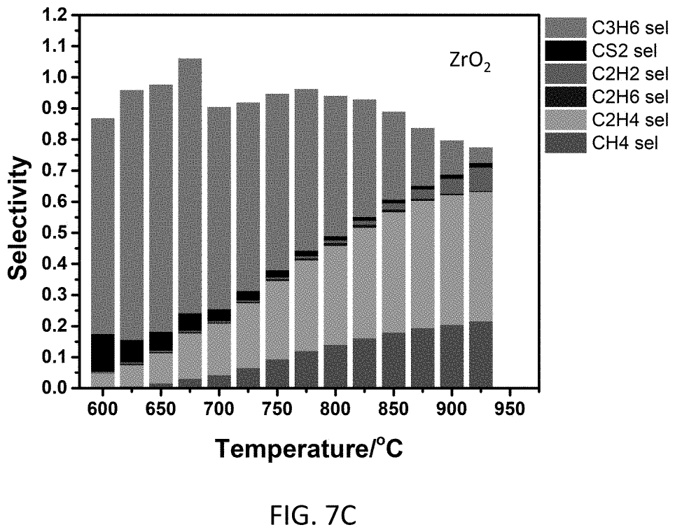

The product distribution as a function of precatalysts and temperature are shown in FIGS. 7A-7C, respectively. These results show that although the precatalyst type doesn't appear to affect the selectivity for C₃H₆, it does shift the product distribution. The formation of CS₂ and acetylene are dependent on the surface. This may indicate a stepwise reaction mechanism, where surface catalyze sequential dehydrogenation and hydrogenolysis.

### Example 4

Additional experiments similar to those described above for Example 3 were conducted, but using a sulfided V/Al₂O₃ catalyst. The sulfided V/Al₂O₃ catalyst was prepared from a precatalyst, vanadium oxide supported on alumina. The VOx supported on alumina precatalyst was prepared by incipient wetness impregnation. Aqueous NH₄VO₃ (>99%, Aldrich) was used as the vanadium oxide precursor. The solution was mixed with the alumina support, and the resulting mixture was dried overnight at 120° C. Then, the samples were calcined in air at 550° C. for 6 h. Prior to the SODHP reaction, the precatalyst, VOx/Al₂O₃, was heated to 600° C. and held for 4-6 h under a gas stream containing 0.28 wt % S₂ and 0.33 wt % H₂S.

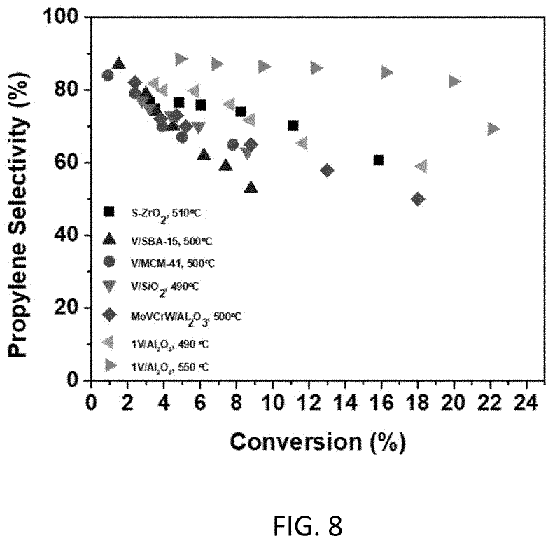

The sulfided catalyst (1V/Al₂O₃, wherein “1” refers to 1.0 weight %) was then exposed to S₂ and C₃H₈ to perform the catalytic SODHP reaction. The sulfur:propane ratio was 0.270, the propane WHSV was 31.7 min⁻¹, and various temperatures were used, including 490° C. and 550° C. as shown in FIG. 8.

In this Example, the precatalyst consists of VOx/Al₂O₃. Specifically, the support consists of the theta-alumina phase, as characterized by powder X-ray diffraction. For low weight loadings of vanadium (here 1.0 weight %), isolated VOx sites form on the surface of the alumina support. Higher weight loadings of vanadium may lead to polymerized VOx or crystalline V₂O₅ supported on alumina. After the sulfidation treatment, the alumina support likely retains its original oxide structure, as no aluminium sulfide is detected after treatment. The VOx sites may be sulfided to vanadium sulfide or oxysulfide sites. Currently, powder X-ray diffraction and X-ray photoelectron spectroscopy characterization suggest the formation of a vanadium sulfide supported on theta-alumina.

Propylene selectivity as a function of propane conversion is shown in FIG. 8. The results using the sulfided 1.0 V/Al₂O₃ catalyst are shown with right pointing (550° C.) and left pointing (490° C.) triangles. Unexpectedly and remarkably, propylene selectivities of nearly 90% are achieved at 550° C. The results for sulfided bulk ZrO₂ catalyst under similar conditions (see Example 3) and other existing metal oxide catalysts using O₂ are also shown. For V/SBA-15, see C. Carrero, et al., Catal. Sci. Technol., 2014, 4, 786-794; for V/MCM-41, see E. V. Kondratenko, et al., J. Catal., 2005, 234, 131-142; for V/SiO₂ see J. T. Grant, et al., ACS Catal., 2015, 5, 5787-5793; and for MoVCrW/Al₂O₃, see E. V. Kondratenko, et al., Catal. Today, 2005, 99, 59-67.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the invention. The embodiments were chosen and described in order to explain the principles of the invention and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

What is claimed is:

1. A method for oxidative dehydrogenation of an alkane, the method comprising exposing a gas comprising an alkane having 2 or more carbons to elemental sulfur vapor at an elevated reaction temperature and for a period of time to convert the alkane to one or more products via oxidative dehydrogenation, the one or more products comprising a primary alkene.

2. The method of claim 1, wherein the exposure and conversion occur in the absence of a catalyst.

3. The method of claim 2, wherein the exposure and conversion occur in the absence of O₂ and in the absence of an oxygen-containing compound in the gas.

4. The method of claim 1, wherein the exposure and conversion occur in the presence of a catalyst.

5. The method of claim 4, wherein the catalyst is formed by exposing a precatalyst to S₂ and H₂S at an elevated temperature and for an activation time.

6. The method of claim 5, further comprising forming the catalyst from the precatalyst, in situ, prior to exposure to the gas comprising the alkane.

7. The method of claim 5, wherein the elevated temperature in is the range of from 600° C. to 1000° C. and the activation time is in the range of from 1 to 10 hours.

8. The method of claim 4, wherein the exposure and conversion occur in the absence of O₂ and in the absence of an oxygen-containing compound in the gas.

9. The method of claim 5, wherein the precatalyst is a supported transition metal oxide.

10. The method of claim 9, wherein the supported transition metal oxide is vanadium oxide on a metal oxide support.

11. The method of claim 10, wherein the metal oxide support is alumina.

12. The method of claim 5, wherein the catalyst is selected from compounds of formula MₓO_{y}S_{z} wherein M is an alkali metal, an alkaline earth metal, or a transition metal, and wherein x>0, y≥0, and z≥0.

13. The method of claim 12, wherein M is vanadium and the catalyst is supported on a metal oxide support.

14. The method of claim 13, wherein the metal oxide support is alumina.

15. The method of claim 1, wherein the alkane is ethane.

16. The method of claim 1, wherein the alkane is propane.

17. The method of claim 16, wherein the exposure and conversion occur in the presence of a catalyst formed by exposing a precatalyst to S₂ and H₂S at an elevated temperature and for an activation time, and wherein the precatalyst is vanadium oxide on an alumina support.

18. The method of claim 1, wherein the alkane is ethane and the method is characterized by a conversion of ethane of at least 99% at 940° C., a selectivity of ethylene of at least 70% at 940° C., or both.

19. The method of claim 1, wherein the alkane is propane and the method is characterized by a selectivity of propylene of at least 80% at 550° C.

20. The method of claim 1, wherein the method is characterized by a conversion of the alkane, a selectivity of primary alkene, or both, that is constant after at least 60 hours at 940° C.
